(19) United States
(12) Patent Application Publication
(10) Pub. No.: US 2012/0276029 A1
(43) Pub. Date: Nov. 1, 2012

(54) INVERSE EMULSION FOR TREATING THE HAIR COMPRISING A PARTICULAR FATTY SUBSTANCE AND AN ALKALINE AGENT

(75) Inventors: Jean-Marc Ascione, Paris (FR); Gautier Deconinck, Saint Gratien (FR); Caroline Goget, Paris (FR); Delphine Allard, Paris (FR)

(73) Assignee: L'OREAL, Paris (FR)

(21) Appl. No.: 13/518,497

(22) PCT Filed: Dec. 15, 2010
(86) PCT No.: PCT/EP2010/069839

§ 371 Date: Jul. 17, 2012

Related U.S. Application Data
(60) Provisional application No. 61/294,933, filed on Jan. 14, 2010.
(60) Provisional application No. 61/294,934, filed on Jan. 14, 2010.
(60) Provisional application No. 61/296,665, filed on Jan. 20, 2010.
(60) Provisional application No. 61/296,671, filed on Jan. 20, 2010.

(30) Foreign Application Priority Data
Dec. 22, 2009 (FR) 0959392
Dec. 22, 2009 (FR) 0959395
Dec. 22, 2009 (FR) 0959397
Dec. 22, 2009 (FR) 0959400

(51) Int. Cl. A61K 8/58; A61Q 5/10; A61K 8/49; A61Q 5/08; A61K 8/41

(52) U.S. Cl. 424/62; 8/406; 8/416; 8/421

(57) ABSTRACT

The subject of the present invention is the use of a W/O emulsion for treating the hair, characterized in that it comprises:
(a) at least 35% by weight of one or more fatty substances chosen from liquid hydrocarbons, liquid fatty esters, liquid silicones and liquid unsaturated fatty alcohols; and
(b) one or more basifying agents.
The present invention also relates to a dyeing or lightening process.

The subject of the present invention is the use for treating the hair, in particular dyeing or lightening, of an emulsion in the form of a water-in-oil inverse emulsion.

Among the methods for dyeing human keratin fibres, such as the hair, mention may be made of oxidation dyeing or permanent dyeing. More particularly, this dyeing method uses one or more oxidation dyes, usually one or more oxidation bases optionally combined with one or more couplers.

In general, oxidation bases are chosen from ortho- or para-phenylenediamines, ortho- or para-aminophenols and heterocyclic compounds. These oxidation bases are colourless or weakly coloured compounds which, when combined with oxidizing products, can give access to coloured species.

The shades obtained with these oxidation bases are often varied by combining them with one or more couplers, the latter being chosen in particular from aromatic meta-diamines, meta-aminophenols, meta-diphenols and certain heterocyclic compounds, such as indole compounds.

The variety of molecules used as oxidation bases and couplers enables a wide range of colours to be obtained.

Direct dyeing or semi-permanent dyeing is also known. The process conventionally used in direct dyeing consists in applying direct dyes to the keratin fibres, said direct dyes being coloured and colouring molecules which have an affinity for the fibres, in leaving them in and then in rinsing them out.

The direct dyes generally used are chosen from nitrobenzene, anthraquinone, nitropyridine, azo, methine, azomethine, xanthene, acridine, azine or triarylmethane direct dyes.

This type of process does not require the use of an oxidizing agent in order to develop the dyeing. However, it is not out of the question to use such an oxidizing agent in order to obtain, with the dyeing, a lightening effect. This is then referred to as direct or semi-permanent dyeing under lightening conditions.

Permanent or else semi-permanent dyeing processes under lightening conditions therefore consist in using, with the dye composition, an aqueous composition comprising at least one oxidizing agent, under alkaline pH conditions in the vast majority of cases. The role of this oxidizing agent is, inter alia, to degrade the melanin in the hair, which, depending on the nature of the oxidizing agent present, results in a more or less pronounced lightening of the fibres. Thus, for a relatively low degree of lightening, the oxidizing agent is generally hydrogen peroxide. When a greater degree of lightening is required, peroxygenated salts, such as persulphates for example, are normally used, in the presence of hydrogen peroxide.

There is a need for satisfactory effectiveness of the lightening and dyeing products, in particular in terms of lightening power or of dyeing strength and/or of selectivity, while lessening the harmful effects associated with the simultaneous presence of alkaline agents and oxidizing agents such as hydrogen peroxide, in particular lessening the degradation of keratin fibres and reducing the unpleasantness associated with the odour of the alkaline agents used, such as aqueous ammonia and amines.

It is therefore sought to increase the effects of the alkaline agents and/or of the oxidizing agents while at the same time having maximum dyeing or lightening effectiveness on keratin fibres.

This aim and others are achieved by the present invention, the subject of which is thus the use of a water-in-oil (W/O) inverse emulsion comprising (a) at least 35% by weight of one or more fatty substances chosen from liquid hydrocarbons, liquid fatty esters, liquid silicones and liquid unsaturated fatty alcohols; and (b) one or more basifying agents for treating the hair.

The present invention also relates to a process for treating human keratin fibres using the inverse emulsion defined above.

A subject of the invention is similarly a two-compartment device comprising, in one, the inverse emulsion defined above and in the other, a composition containing one or more oxidizing agents.

Other characteristics and advantages of the invention will emerge more clearly on reading the description and the examples which follow.

In the text hereinbelow, unless otherwise indicated, the limits of a range of values are included in that range.

The term “fatty substance” is intended to mean an organic compound that is insoluble in water at ordinary temperature (25° C.) and at atmospheric pressure (760 mmHg; i.e. 1.013×10⁵ Pa) (solubility of less than 5%, and preferably less than 1%, even more preferably less than 0.1%). They have in their structure at least one hydrocarbon-based chain containing at least 6 carbon atoms or a sequence of at least two siloxane groups. In addition, the fatty substances are soluble in organic solvents under the same temperature and pressure conditions, for instance chloroform, ethanol or benzene. In the context of the invention, the fatty substances do not comprise a COOH or COO⁻ carboxylic acid function.

The human keratin fibres treated by means of the process according to the invention are preferably the hair.

The water-in-oil inverse emulsions according to the invention are true emulsions, to be distinguished from microemulsions, which are thermodynamically stable systems, unlike true emulsions.

The size of the droplets of the dispersed phase of the emulsions which are of use in the invention is preferably between 10 nm and 100 μm, and preferably between 200 nm and 50 μm.

This is the mean diameter D(3,2) which can be measured in particular using a laser particle sizer.

Preferably, the concentration of fatty substances chosen from liquid hydrocarbons, liquid fatty esters, liquid silicones and liquid unsaturated fatty alcohols in the inverse emulsion can range from 35% to 90%, preferably from 35% to 75%, even more preferably from 35% to 60%, better still from 35% to 55%, relative to the total weight of the emulsion.

The term “liquid hydrocarbon” is intended to mean a hydrocarbon composed only of carbon and hydrogen atoms which is liquid at ordinary temperature (25° C.) and at atmospheric pressure (760 mmHg, i.e. 1.013×10⁵ Pa).

More particularly, the liquid hydrocarbons are chosen from:
C₆-C₁₆ lower alkanes which are linear or branched, or optionally cyclic. Examples that may be mentioned include hexane, undecane, dodecane, tridecane and isoparaffins such as isohexadecane, isododecane and isodecane.
linear or branched hydrocarbons of mineral, animal or synthetic origin, containing more than 16 carbon atoms, such as liquid paraffins, liquid petroleum jelly, polydecenes, hydrogenated polyisobutene such as Parleam® and squalane.

In one preferred variant, the liquid hydrocarbon(s) is (are) chosen from paraffin oils and liquid petroleum jelly.

The term “liquid unsaturated fatty alcohol” is intended to mean a nonglycerolated and nonoxyalkylenated fatty alcohol which has at least one unsaturation and which is liquid at ordinary temperature (25° C.) and at atmospheric pressure (760 mmHg; i.e. 1.013×10⁵ Pa).

Preferably, the liquid unsaturated fatty alcohols of the invention contain from 8 to 30 carbon atoms.

These liquid unsaturated fatty alcohols have, in their structure, at least one double or triple bond. Preferably, the fatty alcohols of the invention have, in their structure, one or more double bonds. When several double bonds are present, there are preferably 2 or 3 of them, and they may be conjugated or non-conjugated.

These fatty alcohols may be linear or branched.

They may optionally comprise, in their structure, at least one aromatic or nonaromatic ring. They are preferably acyclic.

More particularly, the liquid unsaturated fatty alcohols of the invention are chosen from oleyl alcohol, linoleyl alcohol, linolenyl alcohol and undecylenyl alcohol. Oleyl alcohol is most particularly preferred.

The inverse emulsion according to the invention is a water-in-oil (W/O) emulsion comprising at least 30% by weight of one or more fatty substances chosen from silicones which are liquid at 25° C. and at atmospheric pressure.

The term “liquid silicone” is intended to mean an organopolysiloxane which is liquid at ordinary temperature (25° C.) and at atmospheric pressure (760 mmHg; i.e. 1.013×10⁵ Pa).

Preferably, the liquid silicone is chosen from liquid polydialkylsiloxanes, in particular liquid poly-dimethylsiloxanes (PDMSs), and liquid polyorgano-siloxanes comprising at least one aryl group.

These silicones may also be organomodified. The organomodified silicones that can be used in accordance with the invention are liquid silicones as defined above and comprising, in their structure, one or more organofunctional groups attached by means of a hydrocarbon-based group.

The organopolysiloxanes are defined in greater detail in the book by Walter Noll, “Chemistry and Technology of Silicones” (1968), Academie Press. They may be volatile or non-volatile.

When they are volatile, the silicones are more particularly chosen from those which have a boiling point of between 60° C. and 260° C., and even more particularly from:

(i) cyclic polydialkylsiloxanes comprising from 3 to 7, preferably from 4 to 5, silicon atoms. They are, for example, the octamethylcyclotetrasiloxane sold in particular under the name Volatile Silicone® 7207 by Union Carbide or Silbione® 70045 V2 by Rhodia, the decamethylcyclopentasiloxane sold under the name Volatile Silicone® 7158 by Union Carbide, and Silbione® 70045 V5 by Rhodia, the dodecamethylcyclopentasiloxane sold under the name Silsoft 1217 by Momentive Performance Materials, and also mixtures thereof.

Mention may also be made of cyclocopolymers of the dimethylsiloxane/methylalkylsiloxane type, such as Silicone Volatile® FZ 3109 sold by the company Union Carbide, of formula:

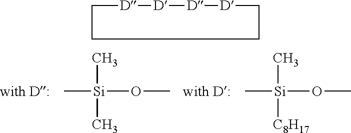

Mention may also be made of the mixtures of cyclic polydialkylsiloxanes with silicon-derived organic compounds, such as the mixture of octamethylcyclo-tetrasiloxane and of tetratrimethylsilylpentaerythritol (50/50) and the mixture of octamethylcyclotetrasiloxane and of oxy-1,1′-bis(2,2,2′,2′,3,3′-hexatrimethylsilyloxy)neopentane;

(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32—Todd & Byers “Volatile Silicone fluids for cosmetics”. The viscosity of the silicones is measured at 25° C. according to standard ASTM 445 Appendix C.

Non-volatile polydialkylsiloxanes may also be used.

These non-volatile silicones are more particularly chosen from polydialkylsiloxanes, among which mention may principally be made of polydimethylsiloxanes having trimethylsilyl end groups.

Among these polydialkylsiloxanes, mention may be made, in a nonlimiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 which has a viscosity of 60 000 mm²/s;
the Viscasil® oils from General Electric and certain oils of the SF series (SF 96, SF 18) from General Electric.

Mention may also be made of polydimethylsiloxanes containing dimethylsilanol end groups, known under the name dimethiconol (CTFA), such as the oils of the 48 series from the company Rhodia.

Among the silicones comprising aryl groups are polydiarylsiloxanes, in particular polydiphenylsiloxanes, and polyalkylarylsiloxanes. By way of example, mention may be made of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

The organomodified liquid silicones may in particular have polyethyleneoxy and/or polypropyleneoxy groups. Mention may thus be made of the silicone KF-6017 proposed by Shin Etsu and the oils Silwet® L 722 and L 77 from the company Union Carbide.

The term “liquid fatty ester” is intended to mean a nonoxyalkylenated ester derived from a fatty acid and/or from a fatty alcohol and which is liquid at ordinary temperature (25° C.) and at atmospheric pressure (760 mmHg; i.e. 1.013×10⁵ Pa).

The liquid fatty esters are preferably liquid esters of saturated or unsaturated, linear or branched C₁-C₂₆ aliphatic mono or polyacids and of saturated or unsaturated, linear or branched C₁-C₂₆ aliphatic mono- or polyalcohols, the total carbon number of the esters being greater than or equal to 10.

Preferably, for the monoalcohol esters, at least one of the alcohol or of the acid from which the esters of the invention are derived is branched.

Among the monoesters of monoacids and of monoalcohols, mention may be made of ethyl palmitate and isopropyl palmitate, alkyl myristates such as isopropyl myristate or ethyl myristate, isocetyl stearate, 2-ethylhexyl isononanoate, isodecyl neopentanoate and isostearyl neopentanoate.

Esters of C₄-C₂₂ dicarboxylic or tricarboxylic acids and of C₁-C₂₂ alcohols and esters of mono-, di- or tricarboxylic acids and of C₄-C₂₆ non sugar di-, tri-, tetra- or pentahydroxy alcohols may also be used.

The following may especially be mentioned: diethyl sebacate; diisopropyl sebacate; di(2-ethylhexyl) sebacate; diisopropyl adipate; di-n-propyl adipate; di(2-ethylhexyl) adipate; diisostearyl adipate; di(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; and diethylene glycol diisononanoate.

The emulsion of the invention may also comprise, as liquid fatty ester, sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which contain at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or non-conjugated carbon-carbon double bonds.

The esters according to this variant may also be chosen from mono-, di-, tri-, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmito-stearate mixed esters.

More particularly, monoesters and diesters and especially sucrose, glucose or methylglucose mono- or dioleates, stearates, behenates, oleopalmitates, linoleates, linolenates and oleostearates are used.

By way of example, mention may be made of the product sold under the name Glucate® DO by the company Amerchol, which is a methylglucose dioleate.

Finally, use may also be made of natural or synthetic esters of mono-, di- or triacids with glycerol.

Among these, mention may be made of plant oils.

As oils of plant origin or synthetic triglycerides that can be used in the emulsion of the invention as liquid fatty esters, mention may be made, for example, of:
triglyceride oils of plant or synthetic origin, such as liquid triglycerides of fatty acids containing from 6 to 30 carbon atoms, for instance the triglycerides of heptanoic acid or octanoic acid, or else, for example, sunflower oil, maize oil, soya oil, marrow oil, grapeseed oil, sesame oil, hazelnut oil, apricot oil, macadamia oil, arara oil, castor oil, avocado oil, caprylic/capric acid triglycerides such as those sold by the company Stearineries Dubois or those sold under the names Miglyol® 810, 812 and 818 by the company Dynamit Nobel, jojoba oil and Shea butter oil.

Preferably, liquid fatty esters derived from monoalcohols will be used as esters according to the invention. Isopropyl myristate or isopropyl palmitate is particularly preferred.

It is recalled that, for the purpose of the invention, the fatty alcohols, esters and acids have more particularly at least one linear or branched, saturated or unsaturated, hydrocarbon-based group containing 6 to 30 carbon atoms, which is optionally substituted, in particular with one or more hydroxyl groups (in particular 1 to 4). If they are unsaturated, these compounds may comprise one to three conjugated or nonconjugated carbon-carbon double bonds.

The emulsions of the invention may comprise one or more other additional fatty substances. More particularly, the additional fatty substances are chosen from solid fatty substances such as waxes.

The (non-silicone) wax(es) is (are) chosen especially from carnauba wax, candelilla wax, esparto grass wax, paraffin wax, ozokerite, plant waxes, for instance olive wax, rice wax, hydrogenated jojoba wax or the absolute waxes of flowers such as the essential wax of blackcurrant flower sold by the company Bertin (France), animal waxes, for instance beeswaxes, or modified beeswaxes (cerabellina); other waxes or waxy raw materials that may be used according to the invention are especially marine waxes such as the product sold by the company Sophim under the reference M82, and waxes of polyethylene or of polyolefins in general.

The silicone gums that can be used in accordance with the invention are especially polydialkylsiloxanes and preferably polydimethylsiloxanes with high number-average molecular masses of between 200 000 and 1 000 000, used alone or as a mixture in a solvent. This solvent can be chosen from volatile silicones, polydimethylsiloxane (PDMS) oils, polyphenyl-methylsiloxane (PPMS) oils, isoparaffins, polyiso-butylenes, methylene chloride, pentane, dodecane and tridecane, or mixtures thereof.

Such mixtures are, for example:
mixtures formed from a polydimethylsiloxane hydroxylated at the chain end, or dimethiconol (CTFA) and from a cyclic polydimethylsiloxane also known as cyclomethicone (CTFA), such as the product Q2 1401 sold by the company Dow Corning;
mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;
mixtures of two PDMSs with different viscosities, and more particularly of a PDMS gum and a PDMS oil, such as the product SF 1236 from the company General Electric. The product SF 1236 is the mixture of an SE 30 gum defined above, having a viscosity of 20 m²/s, and an SF 96 oil, with a viscosity of 5×10⁻⁶ m²/s. This product preferably contains 15% SE 30 gum and 85% SF 96 oil.

The organopolysiloxane resins that can be used in accordance with the invention are crosslinked siloxane systems containing the following units:

R₂SiO_{2/2}, R₃SiO_{1/2}, RSiO_{3/2} and SiO_{4/2}

in which R represents an alkyl containing 1 to 16 carbon atoms. Among these products, the ones that are particularly preferred are those in which R denotes a C₁-C₄ lower alkyl group, more particularly methyl.

Among these resins, mention may be made of the product sold under the name Dow Corning 593 or those sold under the names Silicone Fluid SS 4230 and SS 4267 by the company General Electric, which are silicones of dimethyl/trimethyl siloxane structure.

Mention may also be made of the trimethyl siloxysilicate type resins sold in particular under the names X22-4914, X21-5034 and X21-5037 by the company Shin-Etsu.

The emulsion that is of use according to the invention may have a content of additional fatty substances ranging from 0.1% to 40% by weight, even more preferably from 0.5% to 25% by weight, and better still from 1% to 20% by weight, relative to the weight of the emulsion.

The emulsion of the invention comprises a basifying agent. This agent can be chosen from inorganic or organic alkaline agents or hybrids or mixtures thereof.

The inorganic alkaline agent(s) is (are) preferably chosen from aqueous ammonia, alkaline metal carbonates or bicarbonates, such as sodium or potassium carbonates and sodium or potassium bicarbonates, sodium hydroxide or potassium hydroxide, or mixtures thereof.

The organic alkaline agent(s) is (are) preferably chosen from organic amines of which the pKb at 25° C. is less than 12, and preferably less than 10, even more advantageously less than 6. It should be noted that this is the pKb corresponding to the function of highest basicity.

By way of hybrid compounds, mention may be made of the salts of the abovementioned amines with acids such as carbonic acid or hydrochloric acid.

The organic alkaline agent(s) is (are) for example chosen from alkanolamines, oxyethylenated and/or oxypropylenated ethylene diamines, amino acids and the compounds of formula (V) below:

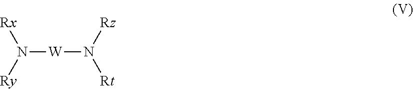

in which W is a C₁-C₆ alkylene residue optionally substituted with a hydroxyl group or a C₁-C₆ alkyl radical; and Rx, Ry, Rz and Rt, which may be identical or different, represent a hydrogen atom or a C₁-C₆ alkyl, C₁-C₆ hydroxyalkyl or C₁-C₆ aminoalkyl radical.

By way of example of such amines, mention may be made of 1,3-diaminopropane, 1,3-diamino-2-propanol, spermine and spermidine.

The term “alkanolamine” is intended to mean an organic amine comprising a primary, secondary or tertiary amine function and one or more linear or branched C₁-C₈ alkyl groups bearing one or more hydroxyl radicals.

Alkanolamines such as mono-, di- or trialkanolamines comprising one to three C₁-C₄ hydroxyalkyl radicals, which may be identical or different, are particularly suitable for implementing the invention.

By way of example, mention may be made of monoethanolamine, diethanolamine, triethanolamine, monoisopropanolamine, diisopropanolamine, N-dimethyl-aminoethanolamine, 2-amino-2-methyl-1-propanol, triisopropanolamine, 2-amino-2-methyl-1,3-propanediol, 3-amino-1,2-propanediol, 3-dimethylamino-1,2-propanediol and trishydroxymethylaminomethane.

More particularly, the amino acids that can be used are of natural or synthetic origin, in their L, D or racemic form, and comprise at least one acid function chosen more particularly from carboxylic, sulphonic, phosphonic or phosphoric acid functions. The amino acids may be in neutral or ionic form.

By way of amino acids that can be used in the present invention, mention may in particular be made of aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine and valine.

Advantageously, the amino acids are basic amino acids comprising an additional amine function optionally included in a ring or in a ureido function.

Such basic amino acids are preferably chosen from those corresponding to formula (VI) below:

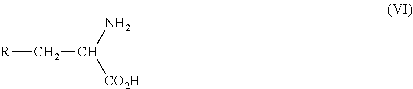

where R denotes a group chosen from:

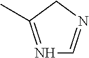

—(CH₂)₃NH₂

—(CH₂)₂NH₂

—(CH₂)₂NHCONH₂

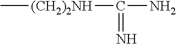

The compounds corresponding to formula (VI) are histidine, lysine, arginine, ornithine and citrulline.

The organic amine can also be chosen from organic amines of heterocyclic type. In addition to the histidine already mentioned in the amino acids, mention may in particular be made of pyridine, piperidine, imidazole, triazole, tetrazole and benzimidazole.

The organic amine can also be chosen from amino acid dipeptides. By way of amino acid dipeptides that can be used in the present invention, mention may in particular be made of carnosine, anserine and baleine.

The organic amine is chosen from compounds comprising a guanidine function. By way of amines of this type that can be used in the present invention, mention may in particular be made, in addition to the arginine already mentioned as an amino acid, of creatine, creatinine, 1,1-dimethylguanidine, 1,1-diethylguanidine, glycocyamine, metformin, agmatine, n-amidinoalanine, 3-guanidinopropionic acid, 4-guanidinobutyric acid and 2-([amino-(imino)methyl]amino)ethane-1-sulphonic acid.

By way of hybrid compounds, mention may in particular be made of guanidine carbonate or monoethanolamine hydrochloride.

The emulsion of the invention preferably contains one or more alkanolamines and/or one or more basic amino acids. According to one embodiment, it contains one or more alkanolamines, for example monoethanolamine.

According to one particular embodiment, the inverse emulsion contains, as alkaline agents, at least one organic amine, preferably at least one alkanolamine. When the emulsion contains several alkaline agents, including an alkanolamine and aqueous ammonia or one of its salts, the organic amine(s) is (are) preferably predominant by weight relative to the amount of ammonia.

According to another embodiment of the present invention, when the inverse emulsion contains aqueous ammonia, it also contains one or more alkanolamines, and the amount by weight of alkanolamine(s) in the emulsion (A) is greater than the amount by weight of ammonia in this same composition.

According to one particular embodiment of the present invention, the emulsion (A) does not contain aqueous ammonia.

Advantageously, the emulsion according to the invention has a content of basifying agent(s) ranging from 0.01% to 30% by weight, preferably from 0.1% to 20% by weight, relative to the weight of the emulsion.

Advantageously, the emulsion according to the invention has a content of basifying agent(s) ranging from 0.01% to 30% by weight, preferably from 0.1% to 20% by weight, relative to the weight of the emulsion.

According to one particular embodiment, the liquid hydrocarbon(s)/basifying agent(s) weight ratio is preferably greater than or equal to 5, better still greater than or equal to 10. Preferably, this ratio varies from 5 to 40, even more preferably from 10 to 25.

According to one variant, the inverse emulsion of the present invention comprises at least one oxidation dye and/or one direct dye.

The oxidation dyes are generally chosen from oxidation bases and couplers.

By way of example, the oxidation bases are chosen from para-phenylenediamines, bisphenylalkylenediamines, para-aminophenols, ortho-aminophenols, heterocyclic bases and their addition salts.

Mention may be made, among para-phenylenediamines, by way of example, of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-(β-hydroxyethyl)-para-phenylenediamine, 2-fluoro-para-phenylenediamine, 2-isopropyl-para-phenylenediamine, N-(β-hydroxypropyl)-para-phenylene-diamine, 2-hydroxymethyl-para-phenylenediamine, N,N-dimethyl-3-methyl-para-phenylenediamine, N,N-(ethyl, β-hydroxyethyl)-para-phenylenediamine, N-(β,γ-dihydroxypropyl)-para-phenylenediamine, N-(4′-amino-phenyl)-para-phenylenediamine, N-phenyl-para-phenylene-diamine, 2-(β-hydroxyethyloxy)-para-phenylenediamine, 2-(β-acetylaminoethyloxy)-para-phenylenediamine, N-(β-methoxyethyl)-para-phenylenediamine, 4-aminophenyl-pyrrolidine, 2-thienyl-para-phenylenediamine, 2-(β-hydroxyethylamino)-5-aminotoluene, 3-hydroxy-1-(4′-aminophenyl)pyrrolidine and their addition salts with an acid.

Among the para-phenylenediamines mentioned above, para-phenylenediamine, para-tolylenediamine, 2-isopropyl-para-phenylenediamine, 2-(β-hydroxyethyl)-para-phenylenediamine, 2-(β-hydroxyethyloxy)-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-(β-acetylaminoethyloxy)-para-phenylenediamine and their addition salts with an acid are particularly preferred.

Mention may be made, among bisphenylalkylene-diamines, by way of example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)-ethylenediamine, N,N′-bis(4-aminophenyl)tetramethylene-diamine, N,N′-bis(β-hydroxyethyl)-N,N′-bis(4-amino-phenyl)tetramethylenediamine, N,N′-bis(4-methylamino-phenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane and their addition salts.

Mention may be made, among para-aminophenols, by way of example, of para-aminophenol, 4-amino-3-methylphenol, 4-amino-3-fluorophenol, 4-amino-3-chlorophenol, 4-amino-3-(hydroxymethyl)phenol, 4-amino-2-methylphenol, 4-amino-2-(hydroxymethyl)phenol, 4-amino-2-(methoxymethyl)phenol, 4-amino-2-(aminomethyl)phenol, 4-amino-2-[(β-hydroxyethyl)aminomethyl]-phenol, 4-amino-2-fluorophenol and their addition salts with an acid.

Mention may be made, among ortho-aminophenols, by way of example, of 2-aminophenol, 2-amino-5-methylphenol, 2-amino-6-methylphenol, 5-acetamido-2-aminophenol and their addition salts.

Mention may be made, among heterocyclic bases, by way of example, of pyridine derivatives, pyrimidine derivatives and pyrazole derivatives.

Mention may be made, among pyridine derivatives, of the compounds described, for example, in Patents GB 1 026 978 and GB 1 153 196, such as 2,5-diamino-pyridine, 2-[(4-methoxyphenyl)amino]-3-aminopyridine, 3,4-diaminopyridine and their addition salts.

Other pyridine oxidation bases of use in the present invention are the 3-aminopyrazolo[1,5-a]-pyridine oxidation bases or their addition salts described, for example, in Patent Application FR 2 801 308. Mention may be made, by way of example, of pyrazolo[1,5-a]pyridin-3-ylamine; 2-(acetylamino)-pyrazolo[1,5-a]pyridin-3-ylamine; 2-(morpholin-4-yl)-pyrazolo[1,5-a]pyridin-3-ylamine; 3-aminopyrazolo-[1,5-a]pyridine-2-carboxylic acid; 2-methoxypyrazolo-[1,5-a]pyridin-3-ylamine; (3-aminopyrazolo[1,5-a]-pyridin-7-yl)methanol; 2-(3-aminopyrazolo[1,5-a]-pyridin-5-yl)ethanol; 2-(3-aminopyrazolo[1,5-a]pyridin-7-yl)ethanol; (3-aminopyrazolo[1,5-a]pyridin-2-yl)methanol; 3,6-diaminopyrazolo[1,5-a]pyridine; 3,4-diaminopyrazolo[1,5-a]pyridine; pyrazolo[1,5-a]-pyridine-3,7-diamine; 7-(morpholin-4-yl)pyrazolo-[1,5-a]pyridin-3-ylamine; pyrazolo[1,5-a]pyridine-3,5-diamine; 5-(morpholin-4-yl)pyrazolo[1,5-a]pyridin-3-ylamine; 2-[(3-aminopyrazolo[1,5-a]pyridin-5-yl)(2-hydroxyethyl)amino]ethanol; 2-[(3-aminopyrazolo[1,5-a]pyridin-7-yl)(2-hydroxyethyl)amino]ethanol; 3-amino-pyrazolo[1,5-a]pyridin-5-ol; 3-aminopyrazolo[1,5-a]pyridin-4-ol; 3-aminopyrazolo[1,5-a]pyridin-6-ol; 3-aminopyrazolo[1,5-a]pyridin-7-ol; and their addition salts.

Mention may be made, among pyrimidine derivatives, of the compounds described, for example, in Patents DE 2 359 399; JP 88-169571; JP 05-63124; EP 0 770 375 or Patent Application WO 96/15765, such as 2,4,5,6-tetraaminopyrimidine, 4-hydroxy-2,5,6-triamino-pyrimidine, 2-hydroxy-4,5,6-triaminopyrimidine, 2,4-dihydroxy-5,6-diaminopyrimidine, 2,5,6-triamino-pyrimidine and their addition salts and their tautomeric forms, when a tautomeric equilibrium exists.

Mention may be made, among pyrazole derivatives, of the compounds described in Patents DE 3 843 892 and DE 4 133 957 and Patent Applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-(tert-butyl)-1-methylpyrazole, 4,5-diamino-1-(tert-butyl)-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-(hydroxymethyl)pyrazole, 4,5-diamino-3-hydroxymethyl-1-methylpyrazole, 4,5-diamino-3-hydroxymethyl-1-isopropylpyrazole, 4,5-diamino-3-methyl-1-isopropyl-pyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-(methylamino)-pyrazole, 3,5-diamino-4-(β-hydroxyethyl)amino-1-methyl-pyrazole and their addition salts. Use may also be made of 4,5-diamino-1-(β-methoxyethyl)pyrazole.

Preferably, use will be made of a 4,5-diaminopyrazole and even more preferably 4,5-diamino-1-(β-hydroxyethyl)pyrazole and/or one of its salts.

Mention may also be made, as pyrazole derivatives, of diamino-N,N-dihydropyrazolopyrazolones, in particular those described in Application FR 2 886 136, such as the following compounds and their addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-di(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-[3-(dimethylamino)pyrrolidin-1-yl]-1,2-diethyl-1,2-dihydropyrazol-3-one or 2,3-diamino-6-hydroxy-6,7-dihydro-1H,5H-pyrazolo[1,2-a]pyrazol-1-one.

Use is preferably made of 2,3-diamino-6,7-dihydro-1H,5H-pyrazolo[1,2-a]pyrazol-1-one and/or one of its salts.

Use will preferably be made, as heterocyclic bases, of 4,5-diamino-1-(β-hydroxyethyl)pyrazole and/or 2,3-diamino-6,7-dihydro-1H,5H-pyrazolo[1,2-a]pyrazol-1-one and/or an addition salt thereof.

The emulsion according to the invention can optionally comprise one or more couplers conventionally used for dyeing keratin fibres.

Mention may in particular be made, among these couplers, of meta-phenylenediamines, meta-aminophenols, meta-diphenols, naphthalene couplers or heterocyclic couplers and their addition salts.

Mention may be made, by way of example, of 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 1H-3-methylpyrazol-5-one, 1-phenyl-3-methylpyrazol-5-one, 2,6-dimethylpyrazolo[1,5-b][1,2,4]triazole, 2,6-dimethyl[3,2-c][1,2,4]triazole, 6-methylpyrazolo[1,5-a]benzimidazole, their addition salts with an acid and their mixtures.

The addition salts of the oxidation bases and couplers are in particular chosen from the addition salts with an acid, such as hydrochlorides, hydrobromides, sulphates, citrates, succinates, tartrates, lactates, tosylates, benzenesulphonates, phosphates and acetates.

The oxidation base(s) each advantageously represent(s) from 0.0001% to 10% by weight, relative to the total weight of the emulsion, and preferably from 0.005% to 5% by weight, relative to the total weight of the emulsion.

The content of coupler(s), if it (they) is (are) present, represents, for each, advantageously from 0.0001% to 10% by weight, relative to the total weight of the emulsion, and preferably from 0.005% to 5% by weight relative to the total weight of the emulsion.

The direct dyes that can be used are, for example, synthetic or natural dyes, chosen from ionic or nonionic, preferably cationic or nonionic, entities.

By way of example of direct dyes which are particularly suitable, mention may be made of nitrobenzene dyes; azo, azomethine or methine direct dyes; azocarbocyanines, such as tetraazacarbocyanines (tetraazapentamethines); quinone, and in particular anthraquinone, naphthoquinone or benzoquinone, direct dyes; azine, xanthene, triarylmethane, indoamine, indigoid, phthalocyanine and porphyrin direct dyes and natural direct dyes, alone or as mixtures. In particular, mention may be made of azo, methine, carbonyl, azine, nitro(hetero)aryl or tri(hetero)arylmethane direct dyes, porphyrins, phthalocyanines and natural direct dyes, alone or as mixtures.

Among the benzene direct dyes that can be used according to the invention, mention may be made, in a nonlimiting manner, of the following compounds:
1,4-diamino-2-nitrobenzene
1-amino-2-nitro-4-β-hydroxyethylaminobenzene
1-amino-2-nitro-4-bis(β-hydroxyethyl)aminobenzene
1,4-bis(β-hydroxyethylamino)-2-nitrobenzene
1-β-hydroxyethylamino-2-nitro-4-bis(β-hydroxyethylamino)benzene
1-β-hydroxyethylamino-2-nitro-4-aminobenzene
1-β-hydroxyethylamino-2-nitro-4-(ethyl)(β-hydroxyethyl) aminobenzene
1-amino-3-methyl-4-β-hydroxyethylamino-6-nitrobenzene
1-amino-2-nitro-4-β-hydroxyethylamino-5-chlorobenzene
1,2-diamino-4-nitrobenzene
1-amino-2-β-hydroxyethylamino-5-nitrobenzene
1,2-bis(β-hydroxyethylamino)-4-nitrobenzene
1-amino-2-tris-(hydroxymethyl)methylamino-5-nitrobenzene
1-hydroxy-2-amino-5-nitrobenzene
1-hydroxy-2-amino-4-nitrobenzene
1-hydroxy-3-nitro-4-aminobenzene
1-hydroxy-2-amino-4,6-dinitrobenzene
1-β-hydroxyethyloxy-2-β-hydroxyethylamino-5-nitrobenzene
1-methoxy-2-β-hydroxyethylamino-5-nitrobenzene
1-β-hydroxyethyloxy-3-methylamino-4-nitrobenzene
1-β,γ-dihydroxypropyloxy-3-methylamino-4-nitrobenzene
1-β-hydroxyethylamino-4-β,γ-dihydroxypropyloxy-2-nitrobenzene
1-β,γ-dihydroxypropylamino-4-trifluoromethyl-2-nitrobenzene
1-β-hydroxyethylamino-4-trifluoromethyl-2-nitrobenzene
1-β-hydroxyethylamino-3-methyl-2-nitrobenzene
1-β-aminoethylamino-5-methoxy-2-nitrobenzene
1-hydroxy-2-chloro-6-ethylamino-4-nitrobenzene
1-hydroxy-2-chloro-6-amino-4-nitrobenzene
1-hydroxy-6-bis(β-hydroxyethyl)amino-3-nitrobenzene
1-β-hydroxyethylamino-2-nitrobenzene
1-hydroxy-4-β-hydroxyethylamino-3-nitrobenzene.

Among the azo, azomethine, methine, or tetraazapentamethine direct dyes that can be used according to the invention, mention may be made of the cationic dyes described in parent applications WO 95/15144, WO 95/01772 and EP 714954; FR 2189006, FR 2285851, FR 2140205, EP 1378544 and EP 1674073.

Mention may be made of the following dyes:

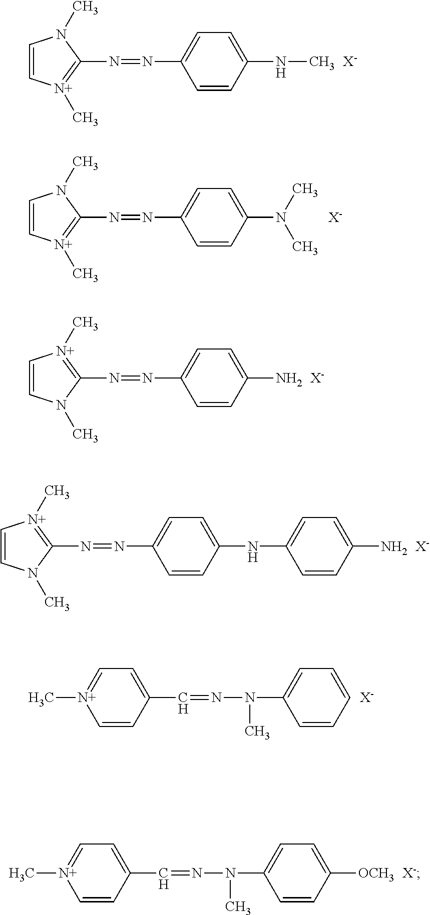

Among the azo direct dyes, mention may also be made of the following dyes described in the Colour Index International, 3rd Edition:
Disperse Red 17
Basic Red 22
Basic Red 76
Basic Yellow 57
Basic Brown 16
Basic Brown 17
Disperse Black 9.

Mention may also be made of 1-(4′-aminodiphenylazo)-2-methyl-4-bis(β-hydroxyethyl) aminobenzene.

Among the quinine direct dyes, mention may be made of the following dyes:
Disperse Red 15
Solvent Violet 13
Disperse Violet 1
Disperse Violet 4
Disperse Blue 1
Disperse Violet 8
Disperse Blue 3
Disperse Red 11
Disperse Blue 7
Basic Blue 22
Disperse Violet 15
Basic Blue 99

and also the following compounds:
1-N-methylmorpholiniumpropylamino-4-hydroxyanthraquinone
1-Aminopropylamino-4-methylaminoanthraquinone
1-Aminopropylaminoanthraquinone
5-β-hydroxyethyl-1,4-diaminoanthraquinone
2-Aminoethylaminoanthraquinone
1,4-bis(β,γ-dihydroxypropylamino)anthraquinone.

Among the azine dyes, mention may be made of the following compounds:
Basic Blue 17
Basic Red 2.

Among the triarylmethane dyes that can be used according to the invention, mention may be made of the following compounds:
Basic Green 1
Basic Violet 3
Basic Violet 14
Basic Blue 7
Basic Blue 26

Among the indoamine dyes that can be used according to the invention, mention may be made of the following compounds:
2-β-hydroxyethlyamino-5-[bis(β-4′-hydroxyethyl)amino]anilino-1,4-benzoquinone
2-β-hydroxyethylamino-5-(2′-methoxy-4′-amino)anilino-1,4-benzoquinone
3-N(2′-Chloro-4′-hydroxy)phenyl-acetylamino-6-methoxy-1,4-benzoquinoneimine
3-N(3′-Chloro-4′-methylamino)phenylureido-6-methyl-1,4-benzoquinoneimine
3-[4′-N-(Ethyl,carbamylmethyl)amino]phenylureido-6-methyl-1,4-benzoquinoneimine.

Among the tetraazapentamethine-type dyes that can be used according to the invention, mention may be made of the following compounds shown in the table below:

| | |
|---|---|
| 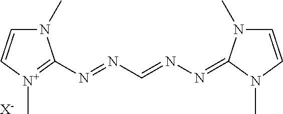 | 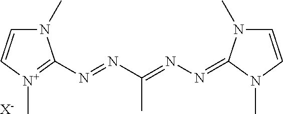 |
| 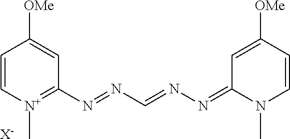 | 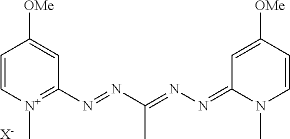 |
| 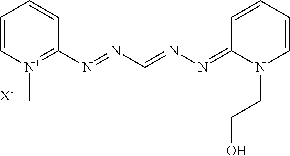 | 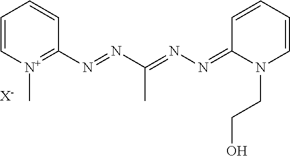 |
| 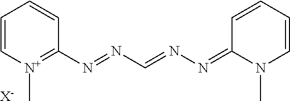 | 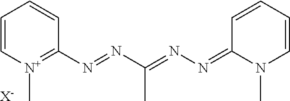 |
| 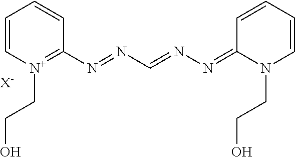 | 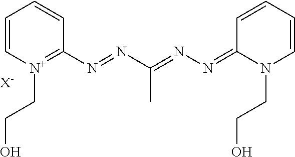 |

X⁻ represents an anion preferably chosen from chloride, iodide, methyl sulphate, ethyl sulphate, acetate and perchlorate.

Among the natural direct dyes that can be used according to the invention, mention may be made of lawsone, juglone, alizarin, purpurin, carminic acid, kermesic acid, purpurogallin, protocatechaldehyde, indigo, isatin, curcumin, spinulosin, apigenidin, hematoxylin, hematein, brasilin, brasilein and orceins. It is also possible to use extracts or decoctions containing these natural dyes, and in particular henna-based poultices or extracts.

The direct dyes may be monochromophoric dyes (i.e. comprising just one dye) or polychromophoric dyes, preferably di- or trichromophoric dyes; it being possible for the chromophores to be identical or different and from the same chemical family or different chemical families. It should be noted that a polychromophoric dye comprises several radicals each derived from a molecule which absorbs in the visible range between 400 and 800 nm. Furthermore, this absorbance by the dye requires neither prior oxidation thereof nor combination with one or more other chemical species(s).

The dye may comprise identical or different chromophores.

By way of example of such dyes, reference may in particular be made to patent applications EP 1637566, EP 1619221, EP 1634926, EP 1619220, EP 1672033, EP 1671954, EP 1671955, EP 1679312, EP 1671951, EP 167952, EP 167971, WO 06/063866, WO 06/063867, WO 06/063868, WO 06/063869, EP 1408919, EP 1377264, EP 1377262, EP 1377261, EP 1377263, EP 1399425, EP 1399117, EP 1416909, EP 1399116 and EP 1671560.

It is also possible to use cationic direct dyes mentioned in patent applications: EP 1006153, which describes dyes comprising two chromophores of anthraquinone type connected by means of a cationic linker arm; EP 1433472, EP 1433474, EP 1433471 and EP 1433473, which describe identical or different dichromophoric dyes connected via a cationic or noncationic linker arm, and also EP 6291333 which describes in particular dyes comprising three chromophores, one of them being an anthraquinone chromophore to which are attached the two chromophores of azo or diazacarbocyanine type or an isomer thereof.

When they are present, the direct dye(s) represent(s) more particularly from 0.0001% to 10% by weight of the total weight of the emulsion, and preferably from 0.005% to 5% by weight.

The emulsion of the invention generally comprises one or more surfactants.

Preferably, the surfactant(s) is (are) chosen from nonionic surfactants or anionic surfactants.

The anionic surfactants are more especially chosen from the salts (in particular alkali metal salts, especially sodium salts, ammonium salts, amine salts, amino alcohol salts or alkaline earth metal salts such as magnesium salts) of the following compounds:
alkyl sulphates, alkyl ether sulphates, alkylamido ether sulphates, alkylaryl polyether sulphates, monoglyceride sulphates;
alkylsulphonates, alkylamidesulphonates, alkylarylsulphonates, α-olefin sulphonates, paraffin sulphonates;
alkyl phosphates, alkyl ether phosphates;
alkylsulphosuccinates, alkyl ether sulphosuccinates, alkylamide sulphosuccinates, alkyl sulphosuccinamates;
alkyl sulphoacetates;
acylsarcosinates, acylisethionates and N-acyl-taurates;
salts of fatty acids such as oleic acid, ricinoleic acid, palmitic acid or stearic acid, coconut oil acid or hydrogenated coconut oil acid;
alkyl-D-galactosiduronic acid salts;
acyllactylates;
salts of polyoxyalkylenated alkyl ether carboxylic acids, of polyoxyalkylenated alkylaryl ether carboxylic acids or of polyoxyalkylenated alkylamido ether carboxylic acids, in particular those containing from 2 to 50 ethylene oxide groups;
and mixtures thereof.

It should be noted that the alkyl or acyl radical of these various compounds advantageously contains from 6 to 24 carbon atoms, and preferably from 8 to 24 carbon atoms, and the aryl radical preferably denotes a phenyl or benzyl group.

The nonionic surfactants are more particularly chosen from monooxyalkylenated or polyoxyalkylenated, monoglycerolated or polyglycerolated nonionic surfactants. The oxyalkylene units are more particularly oxyethylene or oxypropylene units, or a combination thereof, preferably oxyethylene units.

By way of examples of oxyalkylenated nonionic surfactants, mention may be made of:
oxyalkylenated (C₈-C₂₄)alkylphenols,
saturated or unsaturated, linear or branched, oxyalkylenated C₈-C₃₀ alcohols,
saturated or unsaturated, linear or branched, oxyalkylenated C₈-C₃₀ amides,
esters of saturated or unsaturated, linear or branched C₈-C₃₀ acids and of polyethylene glycols,
polyoxyethylenated esters of saturated or unsaturated, linear or branched C₈-C₃₀ acids and of sorbitol,
saturated or unsaturated, oxyethylenated plant oils.

The surfactants contain a number of moles of ethylene oxide and/or of propylene oxide preferably ranging from 1 to 100, even more preferably from 2 to 50 and preferably from 2 to 30.

In accordance with one preferred embodiment of the invention, the oxyalkylenated nonionic surfactants are chosen from oxyethylenated C₈-C₃₀ alcohols comprising from 1 to 100 mol of ethylene oxide.

By way of example of monoglycerolated or polyglycerolated nonionic surfactants, monoglycerolated or polyglycerolated C₈-C₄₀ alcohols are preferably used.

In particular, the monoglycerolated or polyglycerolated C₈-C₄₀ alcohols correspond to the following formula:

RO—[CH₂—CH(CH₂OH)—O]ₘ—H

in which R represents a linear or branched C₈-C₄₀, preferably C₈-C₃₀, alkyl or alkenyl radical, and m represents a number ranging from 1 to 30, and preferably from 1 to 10.

By way of examples of compounds that are suitable in the context of the invention, mention may be made of lauryl alcohol containing 4 mol of glycerol (INCI name: Polyglyceryl-4-Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way as the value of m represents a statistical value, which means that, in a commercial product, several species of polyglycerolated fatty alcohols may coexist in the form of a mixture.

Among the monoglycerolated or polyglycerolated alcohols, it is more particularly preferred to use the C₈-C₁₀ alcohol containing 1 mol of glycerol, the C₁₀-C₁₂ alcohol containing 1 mol of glycerol and the C₁₂ alcohol containing 1.5 mol of glycerol.

Preferably, the surfactant optionally present in the emulsion is a nonionic surfactant.

The surfactant content in the emulsion represents more particularly from 0.1% to 50% by weight, preferably from 0.5% to 30% by weight, relative to the weight of the emulsion.

The emulsion may also contain various adjuvants conventionally used in compositions for dyeing or lightening the hair, such as anionic, cationic, nonionic, amphoteric or zwitterionic polymers, or blends thereof; antioxidants; penetration agents; sequestering agents; fragrances; dispersants; film-forming agents; preservatives; opacifiers.

The above adjuvants are generally present in an amount, for each of them, of between 0.01% and 20% by weight, relative to the weight of the emulsion.

The emulsion may comprise one or more fumed silicas.

The fumed silicas may be obtained by high-temperature hydrolysis of a volatile silicon compound in an oxhydric flame, producing a finely divided silica. This process makes it possible in particular to obtain hydrophilic silicas which have a large number of silanol groups at their surface. Such hydrophilic silicas are, for example, sold under the names Aerosil 130®, Aerosil 200®, Aerosil 255®, Aerosil 300®, and Aerosil 380® by the company Degussa, and Cab-O-Sil HS-5®, CAB-O-Sil EH-5®, Cab-O-Sil LM-130®, Cab-O-Sil MS-55® and Cab-O-Sil M-5® by the company Cabot.

It is possible to chemically modify the surface of the silica by chemical reaction with a view to reducing the number of silanol groups. Silanol groups may in particular be substituted with hydrophobic groups: a hydrophobic silica is then obtained.

The hydrophobic groups may be:
trimethylsiloxyl groups, which are obtained in particular by treating fumed silica in the presence of hexamethyldisilazane. Silicas thus treated are known as “Silica silylate” according to the CTFA (6th Edition, 1995). They are, for example, sold under the references Aerosil R812® by the company Degussa and Cab-O-Sil TS-530® by the company Cabot;
dimethylsilyloxyl or polydimethylsiloxane groups, which are in particular obtained by treating fumed silica in the presence of polydimethylsiloxane or dimethyldichlorosilane. Silicas thus treated are known as “Silica dimethyl silylate” according to the CTFA (6th Edition, 1995). They are, for example, sold under the references Aerosil R972® and Aerosil R974® by the company Degussa and Cab-O-Sil TS-610® and Cab-O-Sil TS-720® by the company Cabot.

The fumed silica preferably has a particle size that may be nanometric to micrometric, for example ranging from approximately 5 to 200 nm.

When it is present, the fumed silica represents from 1% to 30% by weight relative to the weight of the emulsion.

The emulsion may also comprise one or more organic thickeners.

These thickeners may be chosen from fatty acid amides (coconut diethanolamide or monoethanolamide, oxyethylenated alkyl ether carboxylic acid monoethanolamide), polymeric thickeners such as cellulose-based thickeners (hydroxyethylcellulose, hydroxypropylcellulose, carboxymethylcellulose), or guar gum and derivatives thereof (hydroxypropylguar).

The content of organic thickener(s), if it (they) is (are) present, usually ranges from 0.01% to 20% by weight, relative to the weight of the emulsion, preferably from 0.1% to 5% by weight.

The emulsion of the invention comprises water and, optionally, one or more organic solvents.

By way of organic solvent, mention may, for example, be made of linear or branched, preferably saturated, monoalcohols or diols containing 2 to 6 carbon atoms, such as ethyl alcohol, isopropyl alcohol; aromatic alcohols such as benzyl alcohol or phenylethyl alcohol; polyols with more than two hydroxyl functions, such as glycerol; polyol ethers such as, for example, ethylene glycol monomethyl, monoethyl and monobutyl ethers, propylene glycol or ethers thereof, for instance propylene glycol monomethyl ether; and also diethylene glycol alkyl ethers, in particular of C₁-C₄, for instance diethylene glycol monoethyl ether or monobutyl ether, alone or as a mixture.

The organic solvents, when they are present, generally represent between 1% and 40% by weight, relative to the total weight of the emulsion, and preferably between 5% and 30% by weight, relative to the total weight of the emulsion.

In one variant, the concentration of water can range from 5% to 70%, better still from 10% to 50% of the total weight of the emulsion. In another variant, the concentration of the aqueous phase comprising water and the compounds soluble in water at ambient temperature and at atmospheric pressure can range from 10% to 70%, better still from 15% to 50% of the total weight of the emulsion.

The emulsion according to the invention may be in various forms, such as in the form of liquids, milks or creams, or in any other form suitable for dyeing keratin fibres, and in particular human hair.

Preferably, the emulsion is in the form of a milk or a cream.

The pH of the emulsion according to the invention and/or of its aqueous phase is advantageously between 3 and 12, preferably between 5 and 11, and preferentially between 7 and 11, limits inclusive.

It may be adjusted to the desired value by means of acidifying or basifying agents normally used in the dyeing of keratin fibres.

The basifying agents are, for example, those previously described.

Among the acidifying agents, mention may be made, by way of example, of inorganic or organic acids, such as hydrochloric acid or orthophosphoric acid, carboxylic acids such as tartaric acid, citric acid or lactic acid, or sulphonic acids.

The inverse emulsion of the invention is preferably used as a mixture with an oxidizing composition.

The oxidizing composition comprises one or more oxidizing agents.

The oxidizing agent(s) is (are) preferably chosen from the group formed by hydrogen peroxide, urea peroxide, alkali metal bromates or ferricyanides, and persalts such as perborates and persulphates. It is also possible to use, as oxidizing agent, one or more redox enzymes such as laccases, peroxidases and 2-electron oxidoreductases (such as uricase), where appropriate in the presence of the respective donor or cofactor thereof. Hydrogen peroxide is preferred.

The oxidizing composition is preferably an aqueous composition. In particular, it comprises more than 5% by weight of water, preferably more than 10% by weight of water, and even more advantageously more than 20% by weight of water.

It may also comprise one or more organic solvents chosen from those listed previously; these solvents more particularly representing, when they are present, from 1% to 40% by weight, relative to the weight of the oxidizing composition, and preferably from 5% to 30% by weight.

The oxidizing composition also preferably comprises one or more acidifying agents. Among the acidifying agents, mention may be made, by way of example, of inorganic or organic acids, such as hydrochloric acid, orthophosphoric acid or sulphuric acid, carboxylic acids such as acetic acid, tartaric acid, citric acid or lactic acid, and sulphonic acids.

Usually, the pH of the oxidizing composition, when it is aqueous, is less than 7.

Preferably, the oxidizing composition comprises hydrogen peroxide as oxidizing agent, in an aqueous solution, the concentration of which varies, more particularly, from 0.1% to 50%, more particularly between 0.5% and 20%, and even more preferably between 1% and 15% by weight, relative to the weight of the oxidizing composition.

Preferably, the mixture of the inverse emulsion of the invention and of the oxidizing composition is a direct oil-in-water (O/W) emulsion.

This mixture is preferably prepared extemporaneously, i.e. before it is applied to wet or dry human keratin fibres.

After a leave-in time ranging from one minute to one hour, preferably from 5 minutes to 30 minutes.

At the end of the treatment, the human keratin fibres are optionally rinsed with water, optionally washed with a shampoo and then rinsed with water, before being dried or left to dry.

The mixture can be prepared on the hair by applying the W/O emulsion of the invention and the oxidizing composition successively, optionally with a time interval, and without intermediate rinsing. The order of application is unimportant. The leave-in time for each of the two compositions can usually range from one minute to one hour, preferably from 5 minutes to 30 minutes. At the end of the treatment, as in the previous case, the human keratin fibres are optionally rinsed with water, optionally washed with a shampoo and then rinsed with water, before being dried or left to dry.

The temperature during the two processes described above is conventionally between ambient temperature (between 15 and 25° C.) and 80° C., preferably between ambient temperature and 60° C.

According to one particular embodiment, after the emulsion of the invention and the oxidizing agent have been mixed, the amount of fatty substance in the ready-to-use composition is preferably at least 20% by weight of the total weight of the ready-to-use composition, preferably at least 30%.

The invention also relates to a two-compartment device containing, in one, a W/O emulsion as described previously, and in the other, a composition comprising one or more oxidizing agents.

The following examples serve to illustrate the invention without, however, being limiting in nature.

### EXAMPLES

The following inverse emulsion 1 according to the invention is prepared (the amounts are expressed as g %):

| | |
|---|---|
| N,N-BIS(2-HYDROXYETHYL)-P- | 0.124 |
| PHENYLENEDIAMINE SULPHATE•1H₂O | |
| 2-METHYL-1,3-DIHYDROXYBENZENE | 0.276 |
| (2-METHYLRESORCINOL) | |
| 1-BETA-HYDROXYETHYLOXY-2,4- | 0.0138 |
| DIAMINOBENZENE DIHYDROCHLORIDE | |
| 1,3-DIHYDROXYBENZENE (RESORCINOL) | 0.414 |
| 1-METHYL-2,5-DIAMINOBENZENE | 0.744 |
| 1-HYDROXY-3-AMINOBENZENE | 0.124 |
| ETHYL ALCOHOL | 3 |
| MONOETHANOLAMINE | 4 |
| HYDROXYETHYLCELLULOSE (MW: 1 300 000) | 0.44 |
| TERT-BUTYL HYDROQUINONE | 0.09 |
| LIQUID PETROLEUM JELLY | 51 |
| OXYETHYLENATED OLEYL ALCOHOL (10 EO) | 6 |
| SODIUM METABISULPHITE | 0.245 |
| DIETHYLENEDIAMINETETRAACETIC ACID, | 0.26 |
| TETRASODIUM SALT | |
| 2-OCTYLDODECANOL | 9 |
| GLYCEROL | 4.3 |
| WATER | QS 100 |

The oxidizing composition (composition 2) is prepared.

### Composition 2

| | |
|---|---|
| DIETHYLENEDIAMINETETRAACETIC ACID, | 0.11 |
| TETRASODIUM SALT | |
| HYDROGEN PEROXIDE (50% AQUEOUS SOL) | 12 |
| SODIUM STANNATE | 0.04 |
| TETRASODIUM PYROPHOSPHATE | 0.03 |
| TETRAMETHYLHEXAMETHYLENEDIAMINE/1,3- | 0.1 |
| DICHLOROPROPYLENE POLYCONDENSATE (40% | |
| AQUEOUS SOLUTION; HEXADIMETHRINE | |
| CHLORIDE) | |
| POLYDIMETHYLDIALLYLAMMONIUM CHLORIDE | 0.2 |
| (40% AQUEOUS SOLUTION, POLYQUATERNIUM-6) | |
| GLYCEROL | 0.5 |
| CETYLSTEARYL ALCOHOL | 8 |
| OXYETHYLENATED CETYLSTEARYL ALCOHOL | 1.4 |
| (33 EO) | |
| OXYETHYLENATED RAPESEED ACID AMIDE | 1.3 |
| (4 EO) | |
| VITAMIN E; DL-α-TOCOPHEROL | 0.1 |
| PHOSPHORIC ACID | Qs pH |
| | 2.2 |
| WATER | Qs 100 |

### Mode of Application

The two compositions detailed above are mixed weight for weight at the time of use.

The resulting mixture is then applied to locks of natural hair containing 90% grey hairs, in a proportion of 10 g of mixture per 1 g of hair.

The mixture is left in at ambient temperature for 30 minutes.

The hair is then rinsed, washed with a standard shampoo and dried.

Chestnut locks are obtained.

### Example 2

The following inverse emulsion 3 according to the invention is prepared (the amounts are expressed as g %).

| | |
|---|---|
| ETHYL ALCOHOL | 3 |
| MONOETHANOLAMINE | 4 |
| HYDROXYETHYLCELLULOSE (MW: 1 300 000) | 0.44 |
| TERT-BUTYL HYDROQUINONE | 0.09 |
| LIQUID PETROLEUM JELLY | 51 |
| OXYETHYLENATED OLEYL ALCOHOL (10 EO) | 6 |
| SODIUM METABISULPHITE | 0.245 |
| DIETHYLENEDIAMINETETRAACETIC ACID, | 0.26 |
| TETRASODIUM SALT | |
| 2-OCTYLDODECANOL | 9 |
| GLYCEROL | 4.3 |
| WATER | QS 100 |

### Mode of Application

The emulsion 3 detailed above is mixed weight for weight at the time of use with the oxidizing composition 2.

The resulting mixture is then applied to locks of natural chestnut hair, in a proportion of 10 g of mixture per 1 g of hair.

The mixture is left in at ambient temperature for 30 minutes.

The hair is then rinsed, washed with a standard shampoo and dried.

Dark blonde locks are obtained, which reflects a powerful lightening effect.

### Example 3

The following inverse emulsion 3 according to the invention is prepared (the amounts are expressed as g %).

| | |
|---|---|
| N,N-BIS(2-HYDROXYETHYL)-P- | 0.124 |
| PHENYLENEDIAMINE SULPHATE•1H₂O | |
| 2-METHYL-1,3-DIHYDROXYBENZENE | 0.276 |
| (2-METHYLRESORCINOL) | |
| 1-BETA-HYDROXYETHYLOXY-2,4- | 0.0138 |
| DIAMINOBENZENE DIHYDROCHLORIDE | |
| 1,3-DIHYDROXYBENZENE (RESORCINOL) | 0.414 |
| 1-METHYL-2,5-DIAMINOBENZENE | 0.744 |
| 1-HYDROXY-3-AMINOBENZENE | 0.124 |
| ETHYL ALCOHOL | 3 |
| MONOETHANOLAMINE | 4 |
| HYDROXYETHYLCELLULOSE (MW: 1 300 000) | 0.44 |
| TERT-BUTYL HYDROQUINONE | 0.09 |
| OLEYL ALCOHOL | 55 |
| OXYETHYLENATED OLEYL ALCOHOL (10 EO) | 6 |
| SODIUM METABISULPHITE | 0.245 |
| DIETHYLENEDIAMINETETRAACETIC ACID, | 0.26 |
| TETRASODIUM SALT | |
| 2-OCTYLDODECANOL | 9 |
| GLYCEROL | 4.3 |
| WATER | QS 100 |

The inverse emulsion above is mixed weight for weight at the time of use with the oxidizing composition 2 described previously. The resulting mixture is then applied to locks of natural grey hair containing 90% white hairs, in a proportion of 10 g of mixture per 1 g of hair.

The mixture is left in at ambient temperature for 30 minutes.

The hair is then rinsed, washed with a standard shampoo and dried.

Chestnut locks are obtained.

### Example 4

The following inverse emulsion 4 according to the invention is prepared (the amounts are expressed as g %).

| | |
|---|---|
| ETHYL ALCOHOL | 3 |
| MONOETHANOLAMINE | 4 |
| HYDROXYETHYLCELLULOSE (MW: 1 300 000) | 0.44 |
| TERT-BUTYL HYDROQUINONE | 0.09 |
| LIQUID PETROLEUM JELLY | 51 |
| OXYETHYLENATED OLEYL ALCOHOL (10 EO) | 6 |
| SODIUM METABISULPHITE | 0.245 |
| DIETHYLENEDIAMINETETRAACETIC ACID, | 0.26 |
| TETRASODIUM SALT | |
| 2-OCTYLDODECANOL | 9 |
| GLYCEROL | 4.3 |
| WATER | QS 100 |

The inverse emulsion 4 detailed above is mixed weight for weight at the time of use with the oxidizing composition 2 described previously.

The resulting mixture is then applied to locks of natural chestnut hair, in a proportion of 10 g of mixture per 1 g of hair.

The mixture is left in at ambient temperature for 30 minutes.

The hair is then rinsed, washed with a standard shampoo and dried.

Dark blonde locks are obtained, which reflects a powerful lightening effect.

### Example 5

The following inverse emulsions 5A and 5B according to the invention are prepared (the amounts are expressed as g %).

| | EMULSION | EMULSION |
|---|---|---|
| | 5A | 5B |
| N,N-BIS(2-HYDROXYETHYL)-P- | 0.124 | 0.124 |
| PHENYLENEDIAMINE SULPHATE•1H₂O | | |
| 2-METHYL-1,3-DIHYDROXYBENZENE | 0.276 | 0.276 |
| (2-METHYLRESORCINOL) | | |
| 1-BETA-HYDROXYETHYLOXY- | 0.0138 | 0.0138 |
| 2,4-DIAMINOBENZENE | | |
| DIHYDROCHLORIDE | | |
| 1,3-DIHYDROXYBENZENE | 0.414 | 0.414 |
| (RESORCINOL) | | |
| 1-METHYL-2,5-DIAMINOBENZENE | 0.744 | 0.744 |
| 1-HYDROXY-3-AMINOBENZENE | 0.124 | 0.124 |
| ETHYL ALCOHOL | 3 | 3 |
| MONOETHANOLAMINE | 4.5 | 4.5 |
| HYDROXYETHYLCELLULOSE | 0.44 | 0.44 |
| (MW: 1 300 000) | | |
| TERT-BUTYL HYDROQUINONE | 0.09 | 0.09 |
| CYCLOHEXASILOXANE | 48 | — |
| LINEAR POLYDIMETHYLSILOXANE | — | 48 |
| (VISCOSITY: 10 CST) | | |
| OXYETHYLENATED OLEYL | 6 | 6 |
| ALCOHOL (10 EO) | | |
| SODIUM METABISULPHITE | 0.245 | 0.245 |
| DIETHYLENEDIAMINETETRAACETIC | 0.26 | 0.26 |
| ACID, TETRASODIUM SALT | | |
| 2-OCTYLDODECANOL | 9 | 9 |
| GLYCEROL | 4.3 | 4.3 |
| WATER | QS 100 | QS 100 |

The inverse emulsions 5A and 5B are mixed weight for weight with the oxidizing composition 2 described previously, at the time of use.

The resulting mixtures are then applied to locks of natural grey hair containing 90% white hairs, in a proportion of 10 g of mixture per 1 g of hair.

The mixtures are left in at ambient temperature for 30 minutes.

The hair is then rinsed, washed with a standard shampoo and dried.

Chestnut locks are obtained in both cases.

### Example 6

The following inverse emulsions 6A and 6B according to the invention are prepared (the amounts are expressed as g %).

| | EMULSION | EMULSION |
|---|---|---|
| | 6A | 6B |
| ETHYL ALCOHOL | 3 | 3 |
| MONOETHANOLAMINE | 4.5 | 4.5 |
| HYDROXYETHYLCELLULOSE | 0.44 | 0.44 |
| (MW: 1 300 000) | | |
| TERT-BUTYL HYDROQUINONE | 0.09 | 0.09 |
| CYCLOHEXASILOXANE | 48 | — |
| LINEAR POLYDIMETHYLSILOXANE | — | 48 |
| (VISCOSITY: 10 CST) | | |
| OXYETHYLENATED OLEYL | 6 | 6 |
| ALCOHOL (10 EO) | | |
| SODIUM METABISULPHITE | 0.245 | 0.245 |
| DIETHYLENEDIAMINETETRAACETIC | 0.26 | 0.26 |
| ACID, TETRASODIUM SALT | | |
| 2-OCTYLDODECANOL | 9 | 9 |
| GLYCEROL | 4.3 | 4.3 |
| WATER | QS 100 | QS 100 |

### Mode of Application

The inverse emulsions 6A and 6B above are mixed weight for weight at the time of use with the oxidizing composition 2 described previously.

The resulting mixtures are then applied to locks of natural chestnut hair, in a proportion of 10 g of mixture per 1 g of hair.

The mixtures are left in at ambient temperature for 30 minutes.

The hair is then rinsed, washed with a standard shampoo and dried.

Dark blonde locks are obtained in both cases, which reflects a powerful lightening effect.

### Example 7

The following inverse emulsions 7A and 7B according to the invention are prepared (the amounts are expressed as g %).

| | EMULSION | EMULSION |
|---|---|---|
| | 7A | 7B |
| N,N-BIS(2-HYDROXYETHYL)-P- | 0.124 | 0.124 |
| PHENYLENEDIAMINE SULPHATE•1H₂O | | |
| 2-METHYL-1,3-DIHYDROXYBENZENE | 0.276 | 0.276 |
| (2-METHYLRESORCINOL) | | |
| 1-BETA-HYDROXYETHYLOXY-2,4- | 0.0138 | 0.0138 |
| DIAMINOBENZENE | | |
| DIHYDROCHLORIDE | | |
| 1,3-DIHYDROXYBENZENE | 0.414 | 0.414 |
| (RESORCINOL) | | |
| 1-METHYL-2,5-DIAMINOBENZENE | 0.744 | 0.744 |
| 1-HYDROXY-3-AMINOBENZENE | 0.124 | 0.124 |
| ETHYL ALCOHOL | 3 | 3 |
| MONOETHANOLAMINE | 5 | 5 |
| HYDROXYETHYLCELLULOSE | 0.44 | 0.44 |
| (MW: 1 300 000) | | |
| TERT-BUTYL HYDROQUINONE | 0.09 | 0.09 |
| ISOPROPYL MYRISTATE | 53 | |
| SUNFLOWER OIL | | 53 |
| OXYETHYLENATED OLEYL | 6 | 6 |
| ALCOHOL (10 EO) | | |
| SODIUM METABISULPHITE | 0.245 | 0.245 |
| DIETHYLENEDIAMINETETRAACETIC | 0.26 | 0.26 |
| ACID, TETRASODIUM SALT | | |
| 2-OCTYLDODECANOL | 9 | 9 |
| GLYCEROL | 4.3 | 4.3 |
| WATER | QS 100 | QS 100 |

The inverse emulsions 7A and 7B are mixed weight for weight at the time of use with the oxidizing composition 2 described previously.

The resulting mixtures are then applied to locks of natural grey hair containing 90% white hairs, in a proportion of 10 g of mixture per 1 g of hair.

The mixtures are left in at ambient temperature for 30 minutes.

The hair is then rinsed, washed with a standard shampoo and dried.

Chestnut locks are obtained in both cases.

### Example 8

The following inverse emulsions 8A and 8B according to the invention are prepared (the amounts are expressed as g %).

| | EMULSION | EMULSION |
|---|---|---|
| | 8A | 8B |
| ETHYL ALCOHOL | 3 | 3 |
| MONOETHANOLAMINE | 5 | 5 |
| HYDROXYETHYLCELLULOSE | 0.44 | 0.44 |
| (MW: 1 300 000) | | |
| TERT-BUTYL HYDROQUINONE | 0.09 | 0.09 |
| ISOPROPYL MYRISTATE | 53 | |
| SUNFLOWER OIL | | 53 |
| OXYETHYLENATED OLEYL | 6 | 6 |
| ALCOHOL (10 EO) | | |
| SODIUM METABISULPHITE | 0.245 | 0.245 |
| DIETHYLENEDIAMINETETRAACETIC | 0.26 | 0.26 |
| ACID, TETRASODIUM SALT | | |
| 2-OCTYLDODECANOL | 9 | 9 |
| GLYCEROL | 4.3 | 4.3 |
| WATER | QS 100 | QS 100 |

The emulsions 8A and 8B are mixed weight for weight at the time of use with the oxidizing composition 2 described previously.

The resulting mixtures are then applied to locks of natural chestnut hair, in a proportion of 10 g of mixture per 1 g of hair.

The mixtures are left in at ambient temperature for 30 minutes.

The hair is then rinsed, washed with a standard shampoo and dried.

Dark blonde locks are obtained in both cases, which reflects a powerful lightening effect.

1. Use, for treating the hair, of a water-in-oil inverse emulsion comprising:
(a) at least 35% by weight of one or more fatty substances chosen from liquid hydrocarbons, liquid fatty esters, liquid silicones and liquid unsaturated fatty alcohols; and
(b) one or more basifying agents.

2. Use according to the preceding claim, characterized in that the liquid hydrocarbon(s) is (are) chosen from:
linear or branched, optionally cyclic, C₆-C₁₆ lower alkanes,
linear or branched hydrocarbons, of mineral, animal or synthetic origin, of more than 16 carbon atoms.

3. Use according to claim 1 or 2, characterized in that the liquid hydrocarbon(s) is (are) chosen from liquid paraffins and liquid petroleum jelly.

4. Use according to any one of the preceding claims, characterized in that the liquid unsaturated fatty alcohol(s) is (are) linear and comprise(s) one or more double or triple bonds, preferably double bonds.

5. Use according to claim 4, characterized in that the liquid unsaturated fatty alcohol(s) is (are) chosen from oleyl alcohol, linoleyl alcohol, linolenyl alcohol or undecylenyl alcohol, preferably oleyl alcohol.

6. Use according to any one of the preceding claims, characterized in that the liquid silicone(s) is (are) chosen from:
liquid polydialkylsiloxanes, in particular liquid polydimethylsiloxanes (PDMSs),
liquid polyorganosiloxanes comprising at least one aryl group,
liquid organomodified silicones.

7. Use according to claim 6, characterized in that the liquid polydialkylsiloxane(s) is (are) chosen from:
cyclic polydialkylsiloxanes comprising from 3 to 7, preferably from 4 to 5, silicon atoms,
volatile linear polydialkylsiloxanes containing 2 to 9 silicon atoms,
non-volatile liquid polydialkylsiloxanes.

8. Use according to claim 6, characterized in that the liquid polyorganosiloxane(s) comprising at least one aryl group is (are) chosen from polydiarylsiloxanes, preferably polydiphenylsiloxanes, and polyalkylarylsiloxanes.

9. Use according to claim 6, characterized in that the liquid organomodified silicone(s) is (are) chosen from silicones having polyethyleneoxy and/or polypropyleneoxy groups.

10. Use according to any one of the preceding claims, characterized in that the liquid fatty ester(s) is (are) chosen from liquid esters of saturated or unsaturated, linear or branched C₁-C₂₆ aliphatic mono- or polyacids and of saturated or unsaturated, linear or branched C₁-C₂₆ aliphatic mono- or polyalcohols, the total number of carbon atoms of the esters being greater than or equal to 10.

11. Use according to claim 10, characterized in that the liquid fatty ester(s) is (are) chosen from monoalcohol esters, at least one of the alcohol or of the acid from which the esters of the invention are derived being branched.

12. Use according to claim 10, characterized in that the liquid fatty ester(s) is (are):
esters of monoacids and of monoalcohols, chosen from ethyl palmitate and isopropyl palmitate; alkyl myristates such as isopropyl myristate or ethyl myristate; isocetyl stearate; 2-ethylhexyl isononanoate; isodecyl neopentanoate; and isostearyl neopentanoate,
esters of C₄-C₂₂ dicarboxylic or tricarboxylic acids and of C₁-C₂₂ monoalcohols and esters of mono-, di- or tricarboxylic acids and of C₄-C₂₆ non-sugar di-, tri-, tetra- or pentahydroxy alcohols or esters and diesters of sugars and of C₆-C₃₀, preferably C₁₂-C₂₂, fatty acids,
esters of mono, di- or tricarboxylic acids and of glycerol, preferably chosen from plant oils.

13. Use according to any one of the preceding claims, characterized in that the liquid fatty ester(s) is (are) chosen from isopropyl myristate or isopropyl palmitate.

14. Use according to any one of the preceding claims, characterized in that it comprises, as oxidation dyes, one or more oxidation bases chosen from para-phenylenediamines, bisphenylalkylenediamines, para-aminophenols, ortho-aminophenols, heterocyclic bases and addition salts thereof and one or more couplers chosen from meta-phenylenediamines, meta-aminophenols, meta-diphenols, naphthalene couplers, heterocyclic couplers and also addition salts thereof, and/or, as direct dyes, ionic or nonionic, azo, methine, carbonyl, azine, nitro(hetero)aryl or tri(hetero)arylmethane dyes, porphyrins, phthalocyanines and natural direct dyes, alone or as mixtures.

15. Use according to any one of the preceding claims, characterized in that it comprises, as basifying agents, one or more compounds chosen from inorganic alkaline agents such as aqueous ammonia, alkali metal carbonates or bicarbonates, sodium hydroxide or potassium hydroxide, organic alkaline agents such as organic amines of which the pKb at 25° C. is less than 12, and preferably less than 10, even more advantageously less than 6, and hybrid alkaline agents such as the salts of the abovementioned amines with acids such as carbonic acid or hydrochloric acid.

16. Use according to any one of the preceding claims, characterized in that the organic amine is an alkanolamine or a basic amino acid, preferably monoethanolamine.

17. Use according to any one of the preceding claims, characterized in that it comprises one or more surfactants, preferably chosen from non-ionic surfactants or from anionic surfactants, and even more preferably from non-ionic surfactants.

18. Use according to the preceding claim, characterized in that the fatty substance(s) present does (do) not contain a carboxylic acid function.

19. Process for dyeing or lightening human keratin fibres, characterized in that the W/O emulsion as defined in any one of preceding claims 1 to 18 and a composition comprising one or more oxidizing agents are mixed together.

20. Two-compartment device containing, in one, an emulsion as defined previously in claims 1 to 18, and in the other, a composition comprising one or more oxidizing agents.
